(19) United States
(12) Patent Application Publication
(10) Pub. No.: US 2021/0104302 A1
(43) Pub. Date: Apr. 8, 2021

(54) GRAPHICAL USER INTERFACE FOR CHEMICAL TRANSITION STATE CALCULATIONS

(71) Applicant: Schrödinger, Inc., New York, NY (US)

(72) Inventors: Art D. Bochevarov, New York, NY (US); Leif D. Jacobson, Peekskill, NY (US)

(21) Appl. No.: 16/464,588

(22) PCT Filed: Nov. 30, 2017
(86) PCT No.: PCT/US2017/063984

§ 371 Date: May 28, 2019

Related U.S. Application Data
(60) Provisional application No. 62/428,237, filed on Nov. 30, 2016.

(51) Int. Cl. G16C 60/00; G16C 20/80; G16C 20/10; G16C 20/40

(52) U.S. Cl. CPC G16C 60/00; G16C 20/40; G16C 20/10; G16C 20/80

(57) ABSTRACT

A computer-implemented method for finding a transition state for a chemical reaction includes obtaining a graphical representation of one or more reactants of the chemical reaction via a graphical user interface (GUI); (ii) obtaining a graphical representation of one or more reaction products of the chemical reaction via the GUI; (iii) generating an entrance complex and generating an exit complex; (iv) geometrically aligning the entrance complex and the exit complex; (v) calculating an approximate transition state based on the geometrically aligned entrance and exit complexes; (vi) determining the transition state; and (vii) calculating and outputting information about the transition state from the determined transition state.

### TECHNICAL FIELD

This disclosure relates to computational chemistry, and more particularly to using computational chemistry to determine information about transition states of a chemical reaction.

### BACKGROUND

Computational chemistry is a branch of chemistry that uses computer simulation to assist in solving chemical problems. Typically, it uses methods of theoretical chemistry, incorporated into efficient computer programs, to predict the structures and properties of molecules and solids, especially to explore how chemical species interact with each other.

In general, chemical reactions occur by the rearrangement of nuclear configurations from the configuration in one or more reactants to the configuration in one or more reaction products. For polyatomic molecules, there is an enormously large number of possible rearrangement paths that take reactants to reaction products. Reactant molecules that have a lot of energy could follow a path that involves high energy configurations, reactants with less energy will follow a path that involves configurations with lower energy. A complete description of a chemical reaction dynamics would include all these paths. However, such a complete description is challenging—even using current computational methods—because of the need to map out a multidimensional potential energy surface. Instead, a simplified approach, termed the transition state theory, is commonly used.

Typically, an energy barrier separates reactant energy states from reaction product energy states. In many implementations, transition state theory involves finding a path on the multidimensional energy surface linking the reactant state to the product state that avoids the steepest gradients and highest energy states. The highest point on this path is the col, or saddle point that separates the reactant states from the reaction product states. The saddle point is the point of highest energy along the reaction path and is also the point of lowest energy in the direction perpendicular to the reaction path (lowest point of the ridge that separates reactants and products). The saddle point is referred to as the transition state and represents the highest energy configuration of the atomic material as it transitions from the reactants to the reaction products.

Searching for a transition state of a chemical reaction using computational chemistry techniques is often a key component of reactivity and regioselectivity predictions, catalyst design, and mechanistic studies. Finding the transition state, however, can be a long and arduous process that requires many steps and may take days to weeks to complete.

### SUMMARY

The present disclosure relates to an automated transition state search for a chemical reaction. Computational chemistry techniques are used to find transition states and the corresponding transition state barriers using inputs from a simple and intuitive graphical user interface (GUI). The user sketches molecules representing reactants and reaction products of a stoichiometric reaction into appropriate input fields of the GUI. A computer implemented algorithm then analyzes the sketches and applies computational chemistry techniques to return information about a corresponding transition state of the reaction. Due to the simple and intuitive nature of the user interface, the disclosed techniques can be used by experts and non-experts alike.

In general, in a first aspect, the invention features a computer-implemented method for finding a transition state for a chemical reaction. The method includes: (i) obtaining a graphical representation of one or more reactants of the chemical reaction via a graphical user interface (GUI); (ii) obtaining a graphical representation of one or more reaction products of the chemical reaction via the GUI; (iii) generating an entrance complex composed of the one or more reactants based on the graphical representation of the one or more reactants and generating an exit complex composed of the one or more reaction products based on the graphical representation of the one or more reaction products; (iv) geometrically aligning the entrance complex and the exit complex; (v) calculating an approximate transition state based on the geometrically aligned entrance and exit complexes; (vi) determining the transition state based on the approximate transition state; (vii) calculating information about the transition state from the determined transition state; and (viii) outputting the information about the transition state via the GUI.

Implementations of the method can include one or more of the following features. For example, the graphical representations can each include a sketch of a molecule or atom corresponding to each of the one or more reactants and one or more reaction products. The sketch of the molecule can show atoms forming the molecule and chemical bonds between the atoms.

In some embodiments, the method includes performing a conformational search on the reactant(s), transition state(s), and the product(s) and outputting the information about the conformations via the GUI.

The graphical representations can be obtained by having a user input each graphical representations into a corresponding field of the GUI.

The graphical representations of the reactants and reaction products can represent a stoichiometric chemical reaction.

The entrance complex can be generated by arranging the reactants relative to one another in a common reactant coordinate system and the exit complex is generated by arranging the reaction products relative to one another in a common reaction product coordinate system.

Generating the entrance and exit complexes can include identifying each atom and chemical bond in the one or more reactants and identifying each atom and chemical bond in the one or more reaction products.

Generating the entrance and exit complexes can include identifying each atom and chemical bond in the one or more reactants and identifying each atom and chemical bond in the one or more reaction products.

Calculating the approximate transition state can include identifying a corresponding template. The corresponding template can be identified from a plurality of predetermined transition state templates.

Calculating the approximate transition state can include determining a transition path for each atom from the entrance complex to the exit complex and identifying the approximate transition state as an arrangement of the atoms having a maximum energy.

The transition state can be determined from the approximate transition state using an interpolation between different arrangement of the atoms along the transition path from the entrance complex to the exit complex. The interpolation can be performed using a synchronous transit method.

Determining the transition state based on the approximate transition state can include vetting the transition state. The vetting can include vetting a geometry of the transition state. The vetting can include tracing the transition state to the reactants. The vetting can include tracing the transition state to the reaction products.

The information about the transition state can include a structure of the transition state. The information about the transition state can include information about the energetics of the transition state, such as an energy of a transition state barrier corresponding to an energy of a reactant complex and reaction product complex with respect to separated reactants and separated reaction products. The information about the energetics of the transition state can be determined using density functional theory.

The chemical reaction can be: Michael addition, cycloaddition (such as Diels-Alder reaction), Wittig reaction, hydrogen abstraction, hydrogen transfer, oxidative addition, reductive elimination, migratory insertion, alkene insertion, β-Hydrogen elimination, metalation-deprotonation.

In general, in a further aspect, the invention features a non-transient computer readable medium containing program instructions for causing a computer to perform the method of: (i) obtaining a graphical representation of one or more reactants of a chemical reaction via a graphical user interface (GUI); (ii) obtaining a graphical representation of one or more reaction products of the chemical reaction via the GUI; (iii) generating an entrance complex composed of the one or more reactants based on the graphical representation of the one or more reactants and generating an exit complex composed of the one or more reaction products based on the graphical representation of the one or more reaction products; (iv) geometrically aligning the entrance complex and the exit complex; (v) calculating an approximate transition state based on the geometrically aligned entrance and exit complexes; (vi) determining the transition state based on the approximate transition state; (vii) calculating information about the transition state from the determined transition state; and (viii) outputting the information about the transition state via the GUI.

Implementations of the medium can include one or more of the features of the first aspect of the invention.

In general, in another aspect, the invention features a system for determining a transition state for a chemical reaction. The system includes an electronic display, one or more electronic processors in communication with the electronic display, and one or more input devices for allowing a user of the system to interact with the system via a graphical user interface (GUI) presented on the electronic display. The one or more electronic processors are configured to: (i) receive a graphical representation of one or more reactants of the chemical reaction input by the user via the GUI; (ii) receive a graphical representation of one or more reaction products of the chemical reaction input by the user via the GUI; (iii) generate an entrance complex composed of the one or more reactants based on the graphical representation of the one or more reactants and generating an exit complex composed of the one or more reaction products based on the graphical representation of the one or more reaction products; (iv) geometrically align the entrance complex and the exit complex; (v) calculate an approximate transition state based on the geometrically aligned entrance and exit complexes; (vi) determine the transition state based on the approximate transition state; (vii) calculate information about the transition state from the determined transition state; and (viii) output the information about the transition state via the GUI.

Embodiments of the system can include one or more of the features of the prior aspects of the invention.

Among other advantages, the disclosed computer-implemented techniques offer a simple and intuitive setup suitable for non-experts. The techniques can be implemented in ways in which the only input required is structures of the reactants and the products in an elementary reaction step. The techniques can work for a large number of transition state calculations. Libraries of transition state templates can be used to speed up calculates and avoid duplicative calculations. Calculations can be done on many reaction types, in solvent or gas phase, with any DFT functional or basis set.

Generally, disclosed implementations can significantly simplify and speed up the process of performing transition state search and finding the corresponding transition state barriers, and consequently speed up an underlying research project. The disclosed techniques can make transition state search more accessible to non-experts, who would otherwise be deterred from performing transition state search using traditional manual or semi-manual approaches, due to their complexity.

Finding transition states and the corresponding transition state barriers using sketches of chemical reactions as input can simplify investigation of chemical pathways involving multi-step reactions. This can facilitate decision making about, or optimization of such complex chemical processes as organic synthesis pathways involving multiple elementary steps and catalytic cycles in homogeneous catalysis.

Accordingly, the disclosed computer-implemented techniques can provide advantages to other technologies and technical fields. For instance, the disclosed techniques can be used to efficiently determine whether viable reaction pathways exist for generating certain molecules. The ease of use afforded by the disclosed techniques can allow synthetic chemists and/or chemical engineers to utilize computational chemistry techniques without significant technical training in the underlying computational chemistry used to calculate a transition state.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows an input window of a Graphical User Interface (GUI);

FIG. 2 shows an output window of the GUI;

FIG. 3A is a flowchart showing steps in an algorithm for calculating information about a transition state of a chemical reaction using the GUI;

FIG. 3B is a flowchart showing steps in another algorithm for calculating information about a transition state of a chemical reaction using the GUI, including a conformational search;

FIGS. 4A-4E show inputs and outputs generated by applying the disclosed techniques to a variety of different organic reaction types; and

FIG. 5 is a schematic diagram of a computer system suitable for carrying out the operations described in association with the algorithm.

Like reference symbols in the various drawings indicate like elements.

### DETAILED DESCRIPTION

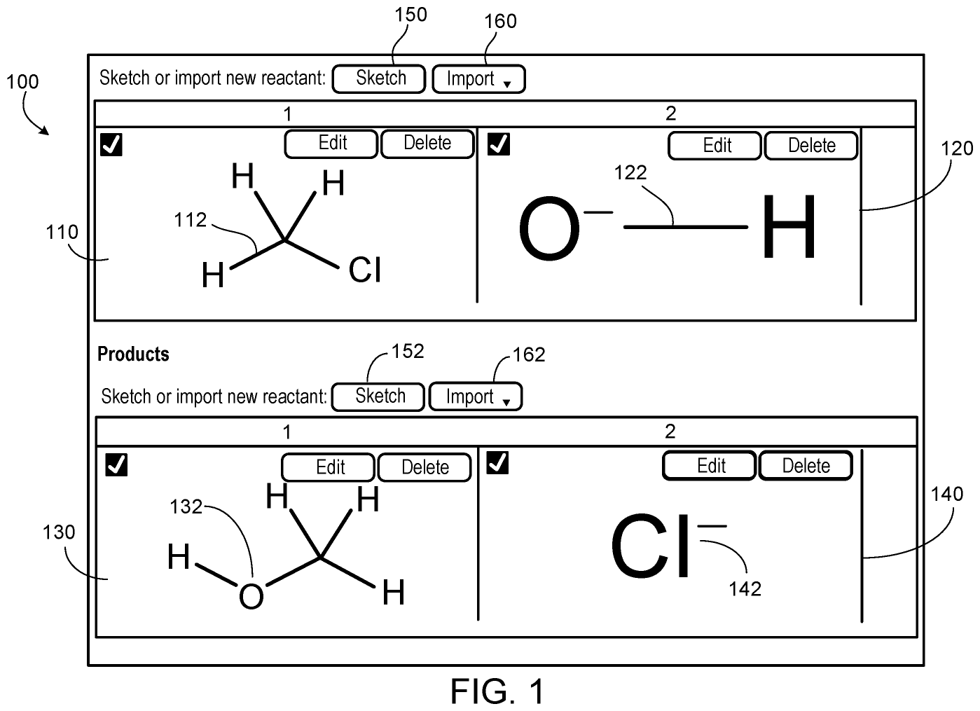

Computational chemistry is used to find transition states and the corresponding transition state barriers using inputs from a simple and intuitive graphical user interface (GUI). Referring to FIG. 1, in an exemplary implementation, the GUI includes an input window 100 that includes input fields 110 and 120 for two reactants and input fields 130 and 140 for two reaction products. The input fields provide a space for the user to sketch reactants and reaction products for a stoichiometric chemical reaction, which are the inputs for a computer algorithm that determines a transition state for the chemical reaction and returns information about the transition state to the user. Details of the algorithm for determining the transition state from the reactants and reaction products are discussed in detail below.

The GUI allows the user to input the reactants and reaction products by sketching representations of the molecules in the respective input fields. For example, as shown in FIG. 1, the sketches can be of conventional two-dimensional structural formulae for each molecule, in which atoms are represented by their corresponding symbol, chemical bonds are shown as lines between the atoms, and charges are indicated by superscript “+” and “−” signs. By way of example, CH₃Cl 112, is the reactant provided in input field 110, OH⁻ 122 is the reactant shown in input field 120, CH₃OH 132 is the reaction product shown in input field 130, and Cl⁻ is the reaction product shown in input field 140. As shown, carbon atoms can be depicted by the vertex between two bonds, e.g., using a skeletal formula.

Other formats can also be used. For example, for simple compounds or individual atoms or ions, empirical chemical formulae may be used. Other common conventions can also be used, such as those conventions used to depict different stereochemistries. For example, relative bond orientations can be sketched using solid wedges or dashed wedges, as per convention. See, e.g., https://en.wikipedia.org/wiki/Structural formula, showing examples of perspective drawings (e.g., Newman projection and sawhorse projection, Cyclohexane conformations, Haworth projections, and Fischer projections) which can be used to sketch reactants and reaction products.

In some embodiments, three dimensional sketches can be used, such as ball and stick sketches.

In general, the GUI permits the user to sketch the reactants and reaction products in a variety of ways. For example, the user can sketch directly into the input field using a touchscreen, mouse, or other drawing device. Alternatively, or additionally, the user can sketch the molecule using a keypad (e.g., for empirical formulae). Palettes or drop down menus can be used to enter special symbols, such as lines with particular orientations, or other bond symbols.

In some embodiments, the GUI can facilitate use of a plug-in (or plug-ins) for formula input. Programs used to sketch molecules include 2DSKETCHER (available from Schrödinger's graphical environment Maestro) and ChemDraw (http://www.cambridgesoft.com/software/overview.aspx), can be used.

In some implementations, the structural formulae for input fields 110, 120, 130, and 140 can be generated using a different software package, saved as a separate file, and the file uploaded by the GUI into the appropriate input field. A variety of file formats are contemplated, including standard graphics files (e.g., JPEG, TIFF, GIF, BMP, etc.), file formats native to chemical graphics programs (e.g., native file formats for ChemDraw are the binary CDX and the preferred XML based CDXML formats), other formats compatible with chemical graphics programs (e.g., ChemDraw can import from, and export to, MOL, SDF, and SKC chemical file formats), and other file formats commonly used for graphics and sketching, such as PPT files. Chemical formulae can also be hand drawn on paper and scanned in for use. Files from commercially-available programs for generating 3D structures can also be used, such as output files generated by PyMol (available from Schrödinger).

Buttons 150, 152, 160, and 162 allow the user to select an input method for each input field.

While the example shown in FIG. 1 depicts a chemical reaction that involves two reactants and two reaction products, more generally, the GUI can provide additional or fewer input windows as the user needs.

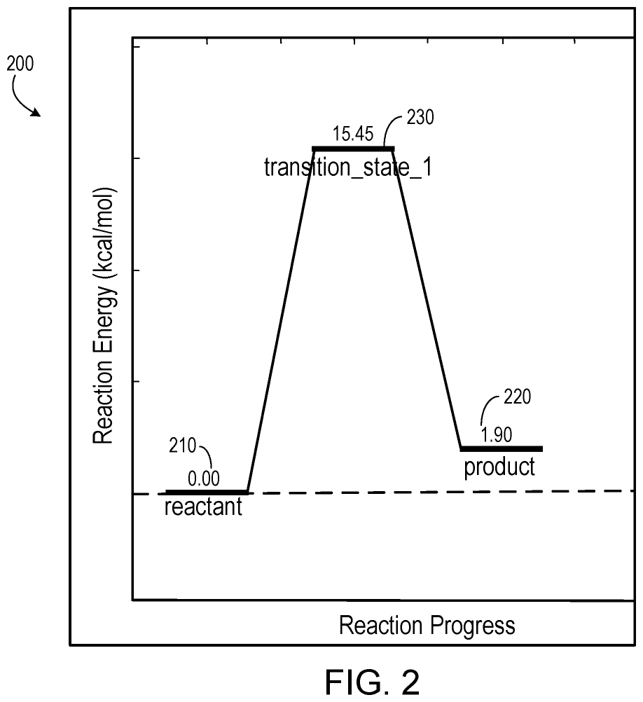

In general, the algorithm used to determine the transition state can return a variety of different information about the transition state to the user, such as a transition state energy. Referring to FIG. 2, for example, in some implementations the GUI returns a graphical representation 200 of the transition state barrier with respect to the reactant and product complexes and with respect to the infinitely separated reactants and products. Here, the reaction progress is shown along the x-axis and reaction energy shown along the y-axis (e.g., in kcal/mol). Here, the energetics of the transition state structure or transition state complex are shown to be 15.45 kcal/mol (at 230) relative to the reactant energy (at 210). The product energy is also shown at 220, in this case 1.90 kcal/mol.

The user can also examine a graphical rendering of the transition state structure. For example, once the transition state is found, the user can examine its 3D structure in a separate window. The transition state vector, together with the associated single imaginary frequency that characterizes the transition state, are also available for inspection. A path from the reactant to the transition state to the product, represented via a sequence of 3D structures, can also be available.

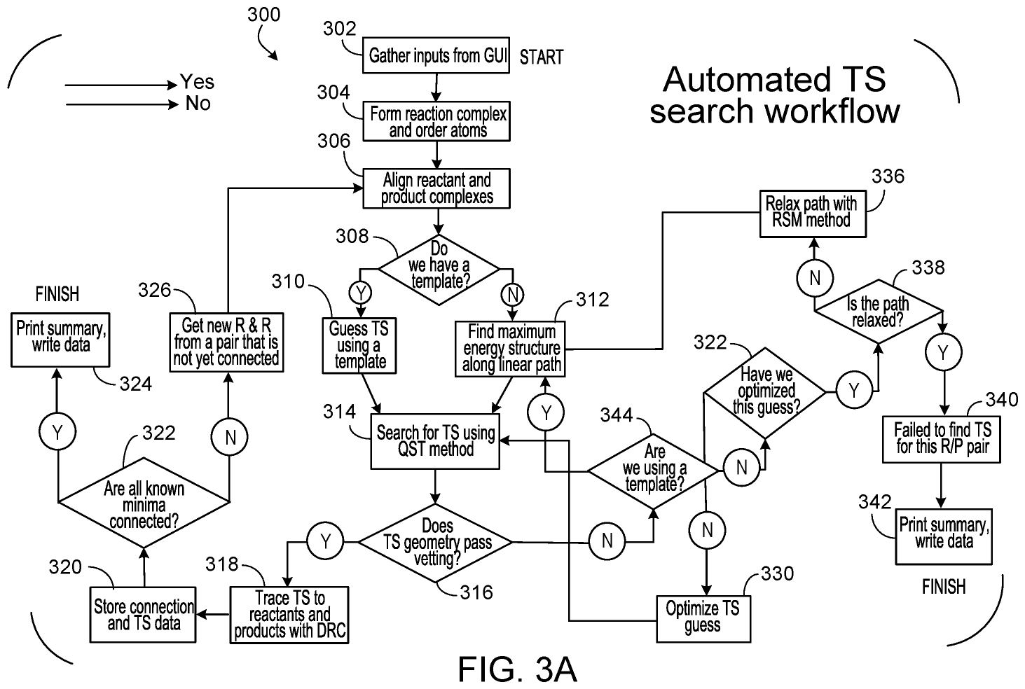

Turning now to details of how a transition state is determined from the inputs to the GUI, FIG. 3 shows a flowchart 300 illustrating sequential operations and computations in an illustrative algorithm.

In a first step 302, the algorithm begins by gathering inputs from the GUI. This step involves identifying the molecule or atom in each input field. Generally, the operations involved in identifying the reactants and reaction products depend on the nature of the input. Where each input is provided as an image file, for example, identification includes performing image recognition on each input to identify each atom, bond, and charge. Conversely, where the input is provided using chemical drawing software, the input may include data that identifies each component of the molecule, or the molecule itself, without involving more fundamental analysis of each image.

The algorithm can also identify bond lengths and bond angles for each bond in each input. Bond lengths and/or bond angles can be determined from first principles or can be identified by looking up values in a database.

Once the molecular structure for each reactant and reaction product is determined, the algorithm proceeds by automatically identifying the covalent chemical bonds that or form in the reaction (as this information can be inferred from the 2-dimensional sketches or the corresponding 3-dimensional representations of the molecules), automatically numbering atoms in the reactants and the products, thus establishing a correspondence between the atoms.

The reaction should be presented stoichiometrically and should ideally represent an elementary reaction step that presumably has only one transition state. The algorithm can check that the reaction is stoichiometric by comparing the composition of the reactants and reaction products to ensure that reaction preserves each atom. In some implementations, the algorithm returns an error to the user if this audit reveals that the reaction, as presented, is not stoichiometric.

In some implementations, the user can provide the total charge and multiplicity (spin) of each entered reactant or product. The total charge and multiplicity can be automatically inferred from 2D representation, but in cases where this is not possible, or where the input can be ambiguous, the user can specify the charge and multiplicity using a dedicated widget in the GUI.

In step 304, the algorithm forms a reaction complex from the analysis of the reactants in step 302 and orders the atoms of the reactants. The algorithm also forms a reaction product complex based on the reaction product analysis from step 302. Generally, the reaction and product complexes are molecular entities formed by loose association of the reactants and reaction products, respectively. Bonding between the constituent species (i.e., the reactants and reaction products) is weaker that in a covalent bond.

In step 306, the algorithm aligns the reactant and product complexes. This involves geometrically reorienting the reactant complex relative to the product complex while maintaining the geometries of the individual complexes to reduce the distance between each atom in the reactant complex and the corresponding atom in the product complex. The algorithm constructs the reactant and product complexes in such a way as to create the minimal path between the reactant and product complexes, avoiding movements of atomic parts that do not directly participate in the reaction. In some implementations, such as those cases where there might be alternative or non-trivial minimal paths, such as in S_{N}2 reactions, such a reaction is recognized, and special rules for creation of its reactant and product complexes, as well as the corresponding reaction path, are applied.

In step 308, the algorithm checks whether a template is available based on the aligned reactant and/or product complexes. For example, the algorithm can compare either or both of the complexes to complexes for which a transition state is known, e.g., by accessing a database of transition state templates. Where the match of the reaction and/or product complexes to those in the database are sufficiently close, the corresponding known transition state can be selected as a template.

In some implementations, for example, the templates are selected by comparing SMARTS patterns (underlying 1D representations) of the entered reactant and the reaction product with those of the saved template. The matching can be performed on several different levels of specificity, and, if several templates match the entered reaction, the most specific template is chosen.

If a template is available, the algorithm—in step 310—guesses a transition state using the template. This involves modifying the template to account for any differences between the structure of the transition state template and the reaction and product complexes. For example, this can include substituting atoms in the template, adding atoms, and/or removing atoms. Ultimately, the template is modified so that the transition state guess consistently accounts for each atom in the reactant and product complexes.

In some embodiments, step 310 can include extracting relevant geometric information from the template and enforcing these geometric parameters on the input reactant structure. For example, in a Michael addition the C-S interatomic distance of the reactant complex is enforced to be the same as that found in the template.

If a template is unavailable, the algorithm—in step 312—generates a guess at a maximum energy structure along a linear path from the aligned reactant and product complexes.

Based on either the template-based guess established in step 310, or the maximum energy structure found in step 312, in step 314 the algorithm searches for the transition state using a synchronous transit method, such as Quadratic Synchronous Transit (QST). QST approximates the reaction path by a parabola instead of a straight line. In some implementations, after an estimate for the transition state is found using a linear path between the reactants and the products, the QST can be generated by minimizing the energy in directions perpendicular to the path, and the QST path can then be searched for an energy maximum. See, e.g., F. Jensen, Introduction to Computational Chemistry, Second Edition, John Wiley & Sons (West Sussex, England).

Alternatively, or in addition, other methods for finding a transition state can be used. For example, one can optimize only the transition state guess, without additional information about the reactant and the product structures. Or, in the coarsest estimation of the transition state energy, one can simply compute the energy of the transition state guess, as well as the corresponding energy barrier, assuming the obtained energy and the barrier roughly representative of those corresponding to the optimized transition state structure.

After finding the transition state, the algorithm vets the transition state geometry in step 316. This can include vetting a geometry of the transition state, such as by checking whether each bond length is within a physically appropriate range and/or whether certain bond angles are within physically appropriate ranges. Alternatively, or additionally, vetting can include tracing the transition state to the reactants and/or reaction products (e.g., using intrinsic reaction coordinates (IRC)).

An additional part of the vetting process can include projecting the transition state vector corresponding to a vibrational frequency on the reaction path. If there is a significant overlap between the vector and the path, the vector is accepted as satisfactory. If the overlap does not exceed a certain threshold, the vector, and possibly the transition state, are rejected as not satisfactory.

If the transition state fails vetting, the algorithm—in step 334—queries whether a template was used. If no template was used, the algorithm repeats step 312, searching for a different maximum energy structure along a linear path between the reactant complex and the product complex. For example, at this stage, the accuracy of the search can be increased.

If a template was used, the algorithm queries whether the guess made using the template in step 310 was optimized. Where no optimization was used, the algorithm—in step 330—performs an optimization of the transition state guess. Step 314, the transition state search, is then performed using the optimized transition state guess.

If the initial transition state guess was optimized, in step 338 the algorithm queries whether the path from the reactant complex to the product complex was relaxed. Here, relaxation refers to commonly-used computational techniques for iteratively finding a saddle point on a mathematical surface.

If the path was not previously a relaxed path, in step 336 the algorithm performs a path relaxation using the RSM method, for example. Alternatively, or additionally, other relaxation methods can be used, for example, nudged elastic band or frozen string method.

However, if the path was previously a relaxed path, the algorithm concludes that no transition state can be found for the reactant/product pair (step 340) and returns this result to the user in step 342.

Referring back to step 316, if the transition state passes vetting, the algorithm traces the transition state structure to the reactants and the reaction products with IRC in step 318.

In step 320, the algorithm then stores the transition state data and data characterizing the connection between the reactants, transition state, and reaction products.

As a final check, the algorithm queries whether all known minima are connected (step 322). For example, the reactant and product complexes, obtained as a result of the IRC step, i.e., by descending from the located transition state along the maximal gradient path in both directions, are compared to the reactant and product complexes generated at step 306 of the algorithm. The comparison can happen on different levels, which can be controlled by the user—the connectivity and the RMSD of the structures can be compared, for example. If the match between the complexes is established, then the transition state is declared to be connected to the corresponding reactants and the products.

If it is determined that there are unconnected known minima, in step 326 the algorithm obtains a new reaction complex and product complex from a pair that is not yet connected and returns these pairs to step 306 for further analysis.

Where all known minima are connected per step 322, the algorithm outputs information about the transition state to the user in step 324, such as shown in FIG. 2 discussed above.

Referring to FIG. 3B, in some implementations an algorithm 400 performs a conformational search 410 on the reactant, the transition state, and the product, if requested by the user. In such cases, information about the conformations can also be included in the printed output in step 324.

In general, information about a transition state is primarily used to understand qualitatively how chemical reactions take place, and can be used to calculate parameters characterizing chemical reactions such as the rate constant, the equilibrium constant, the standard enthalpy of activation ( [symbol] H^{θ}), the standard entropy of activation ( [symbol] S^{θ}), and the standard Gibbs energy of activation ( [symbol] G^{θ}) for a particular reaction.

Information about a transition state can be used to predict reactivity of chemical compounds. For example, information about a transition state can be used to predict reactivity in organic reactions (such as nucleophilic attacks, in particular Michael additions, hydrogen abstractions, hydrogen transfers, cycloadditions, etc) in areas such as organic synthesis and process chemistry. Such predictions allow one to, for example, select optimal pathways in organic synthesis, predict intrinsic reactivity and toxicity of covalent binders in drug design, and investigate mechanistically chemical reaction pathways. Information can also be used to optimize structure of catalysts in homogeneous catalysis.

A chemical reaction can be characterized by a conformational ensemble of reactants, transition states, and products. This conformational ensemble is a result of the flexibility in regions of molecular structures distant from the reaction center. Generally, only the lower energy structures of this ensemble are chemically important in determining the reactivity properties of the input reaction of interest. In order to find the most important reactant(s), transition state(s) and product(s) a conformational search may be performed, if requested by the user. Such a search can use a previously located reactant, transition state and reaction product structures as input seed structures. During the search for low lying transition state structures, the molecular structure of the reacting region of the transition state is typically held fixed while distant regions are allowed to change in order to find low energy conformations. The conformations found to be low in energy are stored and used to compute reactivity properties.

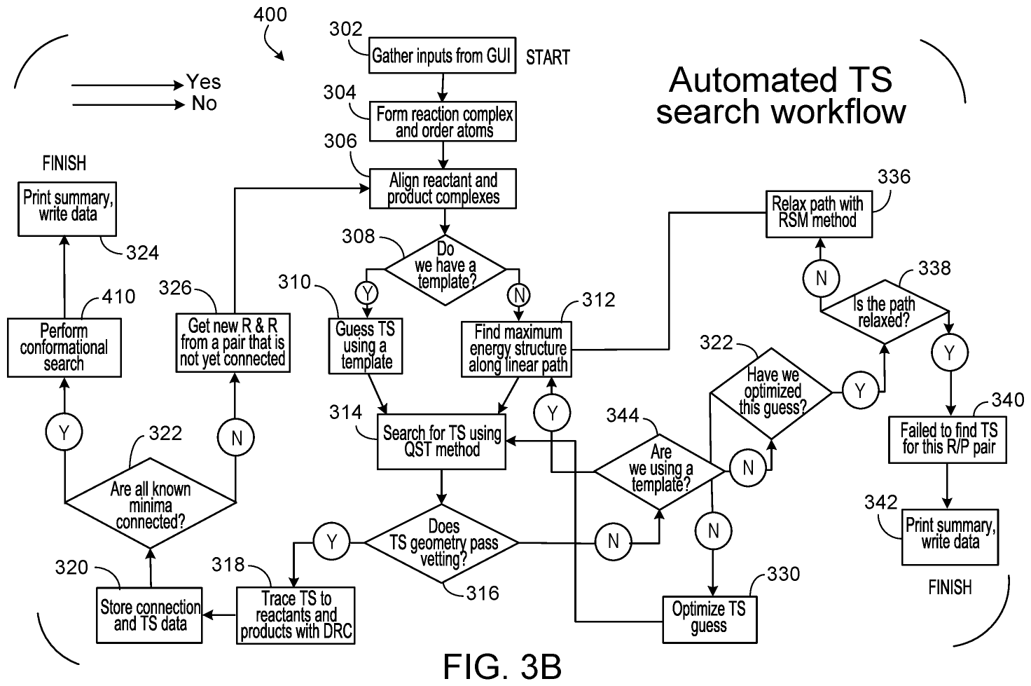

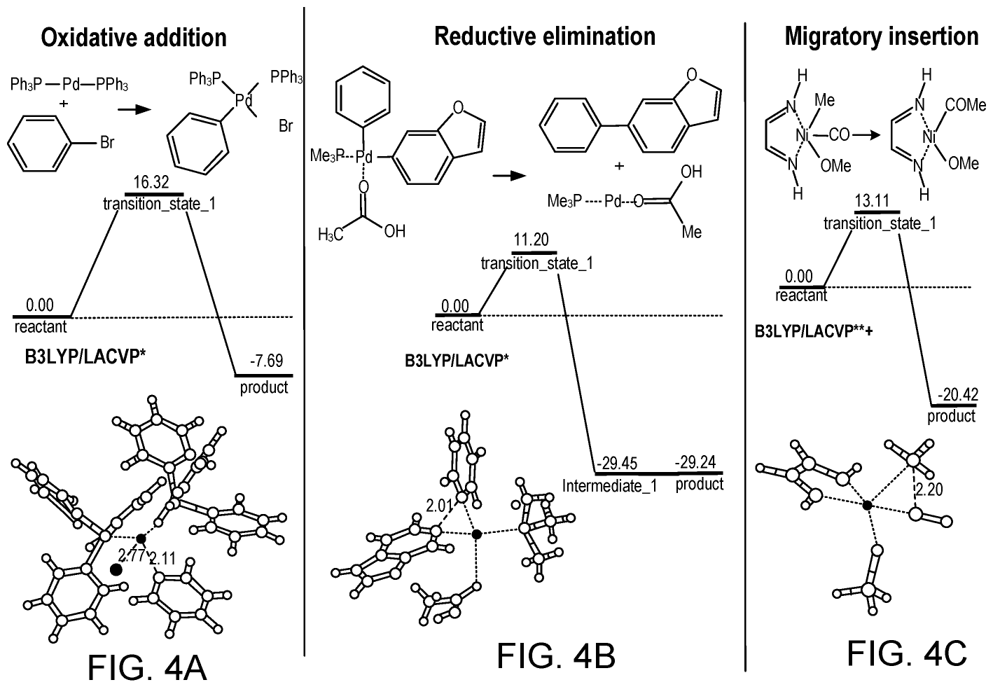

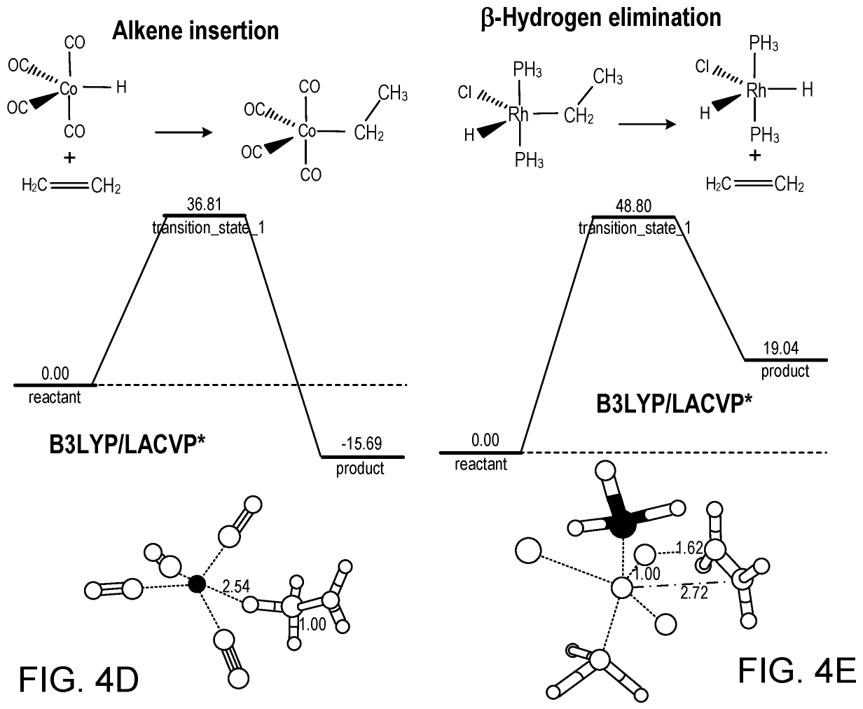

Referring to FIGS. 4A-4E, the disclosed techniques can be also applied to a variety of different metalo-organic reaction types including, for example, oxidative addition (FIG. 4A), reductive elimination (FIG. 4B), migratory insertion (FIG. 4C), alkene insertion (FIG. 4D), and β-hydrogen elimination (FIG. 4E). In each example, the input reactant and reaction product molecules are shown at the top of each figure. A graphical depiction of the transition state energy barrier is shown below the inputs, where the reactant, transition state, and product energies are shown in units of kcal/mol. At the bottom of each figure a ball-and-stick depiction of each transition state is shown, including some bond distances (in Å).

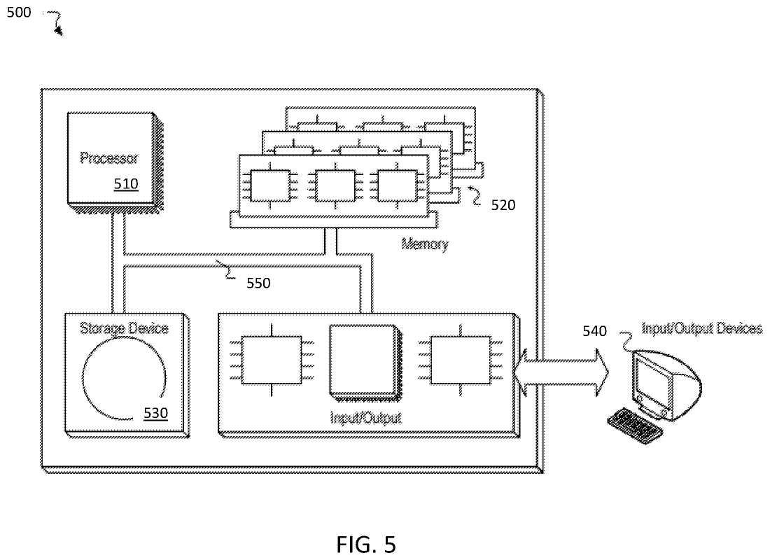

FIG. 5 is a schematic diagram of a computer system 500 suitable for carrying out the operations described in association with any of the computer-implemented methods described previously. In some implementations, computing systems and devices and the functional operations described in this specification can be implemented in digital electronic circuitry, in tangibly-embodied computer software or firmware, in computer hardware, including the structures disclosed in this specification (e.g., system 500) and their structural equivalents, or in combinations of one or more of them. The system 500 is intended to include various forms of digital computers, such as laptops, desktops, workstations, personal digital assistants, servers, blade servers, mainframes, and other appropriate computers, including vehicles installed on base units or pod units of modular vehicles. The system 500 can also include mobile devices, such as personal digital assistants, cellular telephones, smartphones, and other similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 500 includes a processor 510, a memory 520, a storage device 530, and an input/output device 540. Each of the components 510, 520, 530, and 540 are interconnected using a system bus 550. The processor 510 is capable of processing instructions for execution within the system 500. The processor may be designed using any of a number of architectures. For example, the processor 510 may be a CISC (Complex Instruction Set Computers) processor, a RISC (Reduced Instruction Set Computer) processor, or a MISC (Minimal Instruction Set Computer) processor.

In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output device 540.

The memory 520 stores information within the system 500. In one implementation, the memory 520 is a computer-readable medium. In one implementation, the memory 520 is a volatile memory unit. In another implementation, the memory 520 is a non-volatile memory unit.

The storage device 530 is capable of providing mass storage for the system 500. In one implementation, the storage device 530 is a computer-readable medium. In various different implementations, the storage device 530 may be a floppy disk device, a hard disk device, an optical disk device, or a tape device.

The input/output device 540 provides input/output operations for the system 500. In one implementation, the input/output device 540 includes a keyboard and/or pointing device. In another implementation, the input/output device 540 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

A number of embodiments of the invention have been described. Other embodiments are within the scope of the following claims.

1. A computer-implemented method for finding a transition state for a chemical reaction, comprising:
obtaining a graphical representation of one or more reactants of the chemical reaction via a graphical user interface (GUI);
obtaining a graphical representation of one or more reaction products of the chemical reaction via the GUI;
generating an entrance complex composed of the one or more reactants based on the graphical representation of the one or more reactants and generating an exit complex composed of the one or more reaction products based on the graphical representation of the one or more reaction products;
geometrically aligning the entrance complex and the exit complex;
calculating an approximate transition state based on the geometrically aligned entrance and exit complexes;
determining the transition state based on the approximate transition state;
calculating information about the transition state from the determined transition state; and
outputting the information about the transition state via the GUI.

2. The method of claim 1, wherein the graphical representations each comprise a sketch of a molecule or atom corresponding to each of the one or more reactants and one or more reaction products,
wherein the sketch of the molecule shows atoms forming the molecule and chemical bonds between the atoms.

3. (canceled)

4. The method of claim 1, wherein the graphical representations are obtained by having a user input each graphical representations into a corresponding field of the GUI.

5. The method of claim 1, wherein the graphical representations of the reactants and reaction products represent a stoichiometric chemical reaction.

6. The method of claim 1, wherein the entrance complex is generated by arranging the reactants relative to one another in a common reactant coordinate system and the exit complex is generated by arranging the reaction products relative to one another in a common reaction product coordinate system.

7. The method of claim 1, wherein generating the entrance and exit complexes comprises identifying each atom and chemical bond in the one or more reactants and identifying each atom and chemical bond in the one or more reaction products.

8. The method of claim 1, wherein generating the entrance and exit complexes comprises identifying each atom and chemical bond in the one or more reactants and identifying each atom and chemical bond in the one or more reaction products.

9. The method of claim 1, wherein calculating the approximate transition state comprises identifying a corresponding template,
wherein the corresponding template is identified from a plurality of predetermined transition state templates.

10. (canceled)

11. The method of claim 1, wherein calculating the approximate transition state comprises determining a transition path for each atom from the entrance complex to the exit complex and identifying the approximate transition state as an arrangement of the atoms having a maximum energy.

12. The method of claim 1, wherein the transition state is determined from the approximate transition state using an interpolation between different arrangement of the atoms along the transition path from the entrance complex to the exit complex.

13. The method of claim 12, wherein the interpolation is performed using a synchronous transit method.

14. The method of claim 1, wherein determining the transition state based on the approximate transition state comprises vetting the transition state.

15. The method of claim 14, wherein the vetting comprises vetting a geometry of the transition, tracing the transition state to the reactants, and/or tracing the transition state to the reaction products.

16-17. (canceled)

18. The method of claim 1, wherein the information about the transition state comprises a structure of the transition state.

19. The method of claim 1, wherein the information about the transition state comprises information about the energetics of the transition state.

20. The method of claim 19, wherein the information about the energetics of the transition state comprises an energy of a transition state barrier corresponding to an energy of a reactant complex and reaction product complex with respect to separated reactants and separated reaction products.

21. (canceled)

22. The method of claim 1, wherein the chemical reaction is a reaction selected from the group consisting of: oxidative addition, reductive elimination, migratory insertion, alkene insertion, β-Hydrogen elimination, metalation-deprotonation.

23. The method of claim 1, further comprising performing a conformational search on the one or more reactants, the transition state, and/or the one or more reaction products and wherein outputting information about the transition state further comprises outputting information about conformations of the transition state based on the conformational search.

24. (canceled)

25. A non-transient computer readable medium containing program instructions for causing a computer to perform the method of:
obtaining a graphical representation of one or more reactants of a chemical reaction via a graphical user interface (GUI);
obtaining a graphical representation of one or more reaction products of the chemical reaction via the GUI;
generating an entrance complex composed of the one or more reactants based on the graphical representation of the one or more reactants and generating an exit complex composed of the one or more reaction products based on the graphical representation of the one or more reaction products;
geometrically aligning the entrance complex and the exit complex;
calculating an approximate transition state based on the geometrically aligned entrance and exit complexes;
determining the transition state based on the approximate transition state;
calculating information about the transition state from the determined transition state; and
outputting the information about the transition state via the GUI.

26. A system for determining a transition state for a chemical reaction, the system comprising:
an electronic display;
one or more electronic processors in communication with the electronic display; and
one or more input devices for allowing a user of the system to interact with the system via a graphical user interface (GUI) presented on the electronic display,
wherein the one or more electronic processors are configured to:
receive a graphical representation of one or more reactants of the chemical reaction input by the user via the GUI;
receive a graphical representation of one or more reaction products of the chemical reaction input by the user via the GUI;
generate an entrance complex composed of the one or more reactants based on the graphical representation of the one or more reactants and generating an exit complex composed of the one or more reaction products based on the graphical representation of the one or more reaction products;
geometrically align the entrance complex and the exit complex;
calculate an approximate transition state based on the geometrically aligned entrance and exit complexes;
determine the transition state based on the approximate transition state;
calculate information about the transition state from the determined transition state; and
output the information about the transition state via the GUI.
